(19) United States
(12) Patent Application Publication
(10) Pub. No.: US 2019/0022088 A1
(43) Pub. Date: Jan. 24, 2019

(54) USE OF WX-UK1 AND ITS PRODRUG, WX-671, FOR THE TREATMENT OF NON-CANCEROUS MEDICAL CONDITIONS

(71) Applicant: RedHill Biopharma Ltd., Tel-Aviv (IL)

(72) Inventors: Reza Fathi, Oradell, NJ (US); Mark Levitt, Hashmonaim (IL); Terry Plasse, New York, NY (US); Danielle Abramson, Towaco, NJ (US)

(21) Appl. No.: 16/034,815

(22) Filed: Jul. 13, 2018

Related U.S. Application Data
(60) Provisional application No. 62/535,376, filed on Jul. 21, 2017.
(60) Provisional application No. 62/574,449, filed on Oct. 19, 2017.
(60) Provisional application No. 62/589,734, filed on Nov. 22, 2017.

(51) Int. Cl. A61K 31/495; G01N 33/573

(52) U.S. Cl. CPC A61K 31/495; G01N 2800/52; G01N 2333/96433; G01N 33/573

(57) ABSTRACT

The present invention relates to methods of treating an animal having a non-cancerous medical condition that is ameliorated by treatment with a trypsin inhibitor, the method comprising obtaining a biological sample from the animal; testing the sample to obtain a trypsin concentration, wherein, if the trypsin concentration is above the upper limit of the normality, the animal is treated for a suitable period of time by administering to the animal a therapeutically effective amount of a pharmaceutical composition comprising a pharmaceutically-acceptable carrier and the compound N-α-(2,4,6-triisopropylphenylsulfonyl)-3-hydroxyamidino-phenylalanine-4-ethoxycarbonylpiperazide, the stereoisomers, racemic mixtures, metabolite, pharmaceutically acceptable salt, crystal, or any combination thereof. In an embodiment, the non-cancerous medical condition is an inflammatory digestive disease selected from the group consisting of pancreatitis, gastritis, irritable bowel syndrome, and inflammatory bowel disease. In an embodiment, the pharmaceutical composition is in an orally administrable form.

### RELATED APPLICATIONS

This application claims the benefit of and priority to U.S. Provisional Patent Application No. 62/535,376, filed Jul. 21, 2017, U.S. Provisional Patent Application No. 62/574,449, filed Oct. 19, 2017, and U.S. Provisional Patent Application No. 62/589,734, filed Nov. 22, 2017, the entirety of these applications is hereby incorporated herein by reference.

### BACKGROUND

Proteases are important signaling molecules that are involved in numerous vital processes that lead to pathologies, and finding novel therapeutic strategies targeting these proteases is the holy grail of medicine. For small-molecule drugs a key challenge remains the identification and discovery of the molecular targets underlying drug therapeutic effects.

### SUMMARY

According to aspects illustrated herein, high affinity targets for the compound N-α-(2,4,6-triisopropylphenylsulfonyl)-3-amidino-(L)-phenylalanine 4-ethoxycarbonylpiperazide (“WX-UK1”) have been identified with important ramifications in health and disease and which indicates an important usage for WX-UK1 in the treatment of a non-cancerous medical condition. In an embodiment, the non-cancerous medical condition is an inflammatory digestive disease, excluding digestive cancers, that is ameliorated by treatment with a trypsin inhibitor.

According to aspects illustrated herein, a method comprises obtaining a biological sample from an animal having a non-cancerous medical condition; testing the sample to obtain a trypsin concentration, wherein, if the trypsin concentration is above the upper limit of the normality, the animal is treated for a suitable period of time by administering to the animal a therapeutically effective amount of a pharmaceutical composition comprising a pharmaceutically-acceptable carrier and the compound N-α-(2,4,6-triisopropylphenylsulfonyl)-3-hydroxyamidino-phenylalanine-4-ethoxycarbonylpiperazide, the stereoisomers, racemic mixtures, metabolite, pharmaceutically acceptable salt, crystal, or any combination thereof. In an embodiment, the biological sample is selected from the group consisting of blood, serum, urine, saliva, duodenal fluid and intestinal mucosal biopsies. In an embodiment, the non-cancerous medical condition is selected from the group consisting of pancreatitis, gastritis, irritable bowel syndrome, and inflammatory bowel disease. In an embodiment, the non-cancerous medical condition is irritable bowel syndrome. In an embodiment, the irritable bowel syndrome is constipation-predominant irritable bowel syndrome. In an embodiment, the irritable bowel syndrome is diarrhea-predominant irritable bowel syndrome. In an embodiment, the non-cancerous medical condition is inflammatory bowel disease. In an embodiment, the inflammatory bowel disease is Crohn's disease. In an embodiment, the inflammatory bowel disease is ulcerative colitis. In an embodiment, the non-cancerous medical condition is acute pancreatitis. In an embodiment, the pharmaceutical composition is in an orally administrable form. In an embodiment, the compound is present as a sulfate or hydrogen sulfate salt. In an embodiment, the compound is selected from the group consisting of N-α-(2,4,6-triisopropylphenylsulfonyl)-3-hydroxyamidino-(L)phenylalanine-4-ethoxycarbonylpiperazide, N-α-(2,4,6-triisopropylphenylsulfonyl)-3-hydroxyamidino-(D)phenylalanine-4-ethoxycarbonylpiperazide, and N-α-(2,4,6-triisopropylphenylsulfonyl)-3-hydroxyamidino-(D,L)phenylalanine-4-ethoxycarbonylpiperazide, or a physiologically compatible salt thereof. In an embodiment, the compound is N-α-(2,4,6-triisopropylphenylsulfonyl)-3-hydroxyamidino-(L)phenylalanine-4-ethoxycarbonylpiperazide hydrogen sulfate. In an embodiment, the compound is a crystalline form of N-α-(2,4,6-triisopropylphenylsulfonyl)-3-hydroxyamidino-(L)phenylalanine-4-ethoxycarbonylpiperazide or a physiologically compatible salt thereof. In an embodiment, the animal is a human. In an embodiment, the biological sample is tested to obtain a level of trypsin-3. In an embodiment, the biological sample is tested to obtain a level of trypsin-2. In an embodiment, the pharmaceutical composition is co-administered with another drug that is approved for treatment of the non-cancerous medical condition.

According to aspects illustrated herein, a method comprises obtaining a biological sample of duodenal fluid from a human having irritable bowel syndrome; testing the sample to obtain a trypsin-3 concentration, wherein, if the trypsin-3 concentration is above the upper limit of the normality, the human is treated for a suitable period of time by administering to the human a therapeutically effective amount of a pharmaceutical composition comprising a pharmaceutically-acceptable carrier and the compound N-α-(2,4,6-triisopropylphenylsulfonyl)-3-hydroxyamidino-(L)phenylalanine-4-ethoxycarbonylpiperazide hydrogen sulfate.

Until this invention, it was not known that WX-UK1 was a human trypsin-3 inhibitor and that WX-UK1 could be used to treat inflammatory digestive diseases, excluding digestive cancers. Prior to the filing date of this application, WX-UK1 was disclosed for administration in the treatment of, inter alia, cancers. However, WX-UK1 was not known for the treatment of inflammatory non-cancerous digestive diseases including, but not limited to, pancreatitis, gastritis, irritable bowel syndrome, and inflammatory bowel disease. In an embodiment, WX-UK1 and its prodrug WX-671, as detailed below, can be used for the treatment of acute pancreatitis for which there are currently no approved therapies. In an embodiment, WX-UK1 and its prodrug WX-671, as detailed below, can be used for the treatment of irritable bowel syndrome. In an embodiment, WX-UK1 and its prodrug WX-671, as detailed below, can be used for the treatment of inflammatory bowel disease. In an embodiment, the inflammatory bowel disease is Crohn's disease. In an embodiment, the inflammatory bowel disease is ulcerative colitis.

According to aspects illustrated herein, there is disclosed a method of treating pancreatitis in an animal comprising administering to an animal in need of such treatment an effective amount of a compound selected from the group consisting of Formula (I), Formula (II) and Formula (III), or a pharmaceutical acceptable composition containing the compound. In an embodiment, the method further includes obtaining a biological sample from the animal and testing the sample to obtain a level of trypsin. In an embodiment, the biological sample is selected from the group consisting of blood, serum, urine, saliva, duodenal fluid and intestinal mucosal biopsies. In an embodiment, if the mean serum trypsin concentration is above the upper limit of the normality, the animal is treated. In an embodiment, testing the sample to obtain a level of trypsin is carried out using a radioimmunoassay which measures the immunological concentration of trypsin and its pro-enzyme trypsinogen and not their enzymatic biological activity.

According to aspects illustrated herein, there is disclosed use of a compound selected from the group consisting of Formula (I), Formula (II) and Formula (III) in the manufacture of a medicament suitable and intended for the treatment of pancreatitis.

According to aspects illustrated herein, there is disclosed a method of treating irritable bowel syndrome in an animal comprising administering to the mammal in need of such treatment an effective amount of a compound selected from the group consisting of Formula (I), Formula (II) and Formula (III)

, or a pharmaceutical acceptable composition containing the compound. In an embodiment, the irritable bowel syndrome is irritable bowel syndrome with diarrhea. In an embodiment, the irritable bowel syndrome is irritable bowel syndrome with constipation. In an embodiment, the method further includes determining if an animal is in need of such treatment comprising obtaining an intestinal mucosal biopsy sample from the animal and testing the sample to obtain a level of gene expression of PRSS3 (trypsin-3 precursor), wherein, if PRSS3 mRNA is significantly upregulated as compared with a control value from healthy samples, the animal is subjected to treatment by administering an effective amount of either a compound of either formula (I) or formula (II). In an embodiment, testing the sample to obtain a level of gene expression of PRSS3 includes detecting the presence of a PRSS3 transcript. In an embodiment, testing the sample includes in situ zymography.

According to aspects illustrated herein, there is disclosed use of a compound selected from the group consisting of Formula (I), Formula (II) and Formula (III) in the manufacture of a medicament suitable and intended for the treatment of irritable bowel syndrome. In an embodiment, the irritable bowel syndrome is irritable bowel syndrome with diarrhea. In an embodiment, the irritable bowel syndrome is irritable bowel syndrome with constipation.

According to aspects illustrated herein, high affinity targets for the compound N-α-(2,4,6-triisopropylphenylsulfonyl)-3-amidino-(L)-phenylalanine 4-ethoxycarbonylpiperazide (“WX-UK1”) have been identified with important ramifications in health and disease and which indicates an important usage for WX-UK1 in the treatment of human inflammatory lung diseases, excluding lung cancer.

Until this invention, it was not known that WX-UK1 was a human trypsin-2 inhibitor and that WX-UK1 could be used to treat inflammatory lung diseases, excluding lung cancer. Prior to the filing date of this application, WX-UK1 was disclosed for administration in the treatment of, inter alia, cancers. However, WX-UK1 was not known for the treatment of inflammatory lung diseases including, but not limited to, acute lung injury, acute respiratory distress syndrome (ARDS), chronic obstructive pulmonary disease (COPD), emphysema and non-tuberculosis mycobacteria (NTM). In an embodiment, WX-UK1 and its prodrug WX-671, as detailed below, can be used for the treatment of COPD in which there is an unmet medical need.

According to aspects illustrated herein, there is disclosed a method of treating lung disease in an animal comprising administering to an animal in need of such treatment an effective amount of a compound selected from the group consisting of Formula (I), Formula (II) and Formula (III), or a pharmaceutical acceptable composition containing the compound

In an embodiment, the method further includes determining if an animal is in need of such treatment comprising obtaining a lung tissue sample from the animal and testing the tissue sample to obtain a level of gene expression of PRSS2 (trypsin-2 precursor), wherein, if PRSS2 mRNA is significantly upregulated as compared with a control value from healthy lung tissue, the animal is subjected to treatment by administering an effective amount of either a compound of either Formula (I), Formula (II) or Formula (III). In an embodiment, testing the tissue sample to obtain a level of gene expression of PRSS2 includes detecting the presence of a PRSS2 transcript. In an embodiment, testing the tissue sample includes in situ zymography.

According to aspects illustrated herein, there is disclosed use of a compound selected from the group consisting of Formula (I), Formula (II) and Formula (III) in the manufacture of a medicament suitable and intended for the treatment of lung injury.

According to aspects illustrated herein, there is disclosed a method of treating alpha-1 antitrypsin deficiency in an animal, comprising administering to an animal in need of such treatment an effective amount of a compound selected from the group consisting of Formula (I), Formula (II) and Formula (III) or a pharmaceutical acceptable composition containing the compound. In an embodiment, the method further includes determining if the animal is in need of such treatment comprising obtaining a biological sample from the animal and testing the sample to obtain a concentration of alpha-1 antitrypsin. In an embodiment, the biological sample is selected from the group consisting of blood, serum, urine, saliva, duodenal fluid and intestinal mucosal biopsies. In an embodiment, if the mean serum alpha-1 antitrypsin concentration is below the lower limit of the normality, the animal is subjected to treatment by administering an effective amount of either a compound of Formula (I), Formula (II) or Formula (III). In an embodiment, testing the sample to obtain a level of alpha-1 antitrypsin is carried out using a radioimmunoassay which measures the immunological concentration of alpha-1 antitrypsin and not the enzymatic biological activity.

According to aspects illustrated herein, there is disclosed use of a compound selected from the group consisting of Formula (I), Formula (II) and Formula (III) in the manufacture of a medicament suitable and intended for the treatment of alpha-1 antitrypsin deficiency.

A method of treating inflammatory bowel disease in an animal comprises orally administering to an animal in need of such treatment an effective amount of a compound of Formula (III) or a pharmaceutical acceptable composition containing either entity.

A method of treating irritable bowel syndrome in an animal comprises orally administering to an animal in need of such treatment an effective amount of a compound of Formula (III) or a pharmaceutical acceptable composition containing either entity. In an embodiment, the compound is administered orally.

A method of treating pancreatitis in an animal comprises administering to an animal in need of such treatment an effective amount of a compound of Formula (III) or a pharmaceutical acceptable composition containing either entity. In an embodiment, the compound is administered orally.

A method of treating lung disease in an animal comprises administering to an animal in need of such treatment an effective amount of a compound of Formula (III) or a pharmaceutical acceptable composition containing either entity. In an embodiment, the compound is administered orally.

Provided herein also are kits which, when used by the medical practitioner, can simplify the administration of appropriate amounts of dosage forms of the present invention to a patient. In certain embodiments, the kit provided herein includes at least one container having dosage forms of WX-671 provided herein and a label indicating how to use the dosage forms to treat a given non-cancerous medical condition. In an embodiment, a dosage form is a solid dosage form prepared for oral administration. In an embodiment, a dosage form is a dosage form prepared for inhaled administration.

Citation or identification of any reference in this section or any other section of this application shall not be construed as an admission that such reference is available as prior art for the present application.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows reductive conversion of the prodrug WX-671 to pharmacologically active WX-UK1.

FIG. 2 is a schematic representation of the process used to identify WX-UK1 targets.

FIG. 3 shows the structural segments on bovine trypsin in and around the active site of WX-UK1 are primary predictive determinants of WX-UK1 binding.

FIG. 4 is a graph of UK1 activity vs human trypsin-1 inhibitor concentration.

FIG. 5 is a graph of UK1 activity vs human trypsin-2 inhibitor concentration.

FIG. 6 is a graph of UK1 activity vs human trypsin-3 inhibitor concentration.

FIG. 7 is a graph of UK1 activity vs human matriptase (catalytic domain) inhibitor concentration.

FIG. 8 is a schematic showing the experimental setup of a surface plasmon resonance (SPR) experiment in which the ligand of interest (human uPA) is immobilized on the surface of a sensor chip and solutions with different concentrations of WX-UK1 flows over it and the interaction of the solutions to the human uPA is characterized.

FIG. 9 shows binding kinetics of WX-UK1 binding to human uPA.

FIG. 10 is a schematic showing the experimental setup of a SPR experiment in which the ligand of interest is immobilized on the surface of a sensor chip and solutions with different concentrations of WX-UK1 flows over it and the interaction of the solutions to the human uPA is characterized. In an embodiment, the ligand of interest is selected from one of human trypsin-1, human trypsin-3 or human MT-SP1/matriptase.

FIG. 11 shows binding kinetics of WX-UK1 binding to trypsin-1.

FIG. 12 shows binding kinetics of WX-UK1 binding to human trypsin-3.

FIG. 13 shows binding kinetics of WX-UK1 binding to human MT-SP1/matriptase.

### DETAILED DESCRIPTION

### Definitions

A “patient” refers to any animal, such as a primate, such as a human. Any animal can be treated using the methods and composition of the present invention. WX-UK1 and WX-671 determined to be effective for the prevention or treatment of disease or disorders in animals, e.g., rodents, dogs, and monkeys, may also be useful in treatment of diseases in humans. Those skilled in the art of treating diseases in humans will know, based upon data obtained in animal studies, the dosage and route of administration of the compounds to humans. For veterinary use, a compound of the present invention or a non-toxic salt thereof is administered as a suitably acceptable formulation in accordance with normal veterinary practice and the veterinary surgeon will determine the dosing regimen and route of administration which will be most appropriate for a particular animal.

As used herein, the term “a suitable period of time” refers to the period of time starting when a subject begins treatment for a disease using a method of the present disclosure, throughout the treatment, and up until when the subject stops treatment. In an embodiment, a suitable period of time is one (1) week. In an embodiment, a suitable period of time is between one (1) week and two (2) weeks. In an embodiment, a suitable period of time is two (2) weeks. In an embodiment, a suitable period of time is between two (2) weeks and three (3) weeks. In an embodiment, a suitable period of time is three (3) weeks. In an embodiment, a suitable period of time is between three (3) weeks and four (4) weeks. In an embodiment, a suitable period of time is four (4) weeks. In an embodiment, a suitable period of time is between four (4) weeks and five (5) weeks. In an embodiment, a suitable period of time is five (5) weeks. In an embodiment, a suitable period of time is between five (5) weeks and six (6) weeks. In an embodiment, a suitable period of time is six (6) weeks. In an embodiment, a suitable period of time is between six (6) weeks and seven (7) weeks. In an embodiment, a suitable period of time is seven (7) weeks. In an embodiment, a suitable period of time is between seven (7) weeks and eight (8) weeks. In an embodiment, a suitable period of time is eight (8) weeks. In an embodiment, a suitable period of time is between 1 (1) week and two (2) years. In an embodiment, a suitable period of time is beyond two (2) years. Ultimately, the prescribers will decide the appropriate suitable period of time.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

By an “effective amount” or “efficacious amount” or “a therapeutically effective amount” or “therapeutically effective dose” is meant the amount of WX-UK1 or WX-671, which inhibits, totally or partially, the progression of the non-cancerous medical condition or alleviates, at least partially, one or more symptoms of the non-cancerous medical condition. A therapeutically effective amount can also be an amount which is prophylactically effective. The amount which is therapeutically effective will depend upon the patient's size and gender, the non-cancerous medical condition to be treated, the severity of the disease and the result sought. In an embodiment, a therapeutically effective dose refers to that amount of WX-UK1 or WX-671 that results in amelioration of symptoms in a patient. For a given patient, a therapeutically effective amount may be determined by methods known to those of skill in the art. A sufficient effective amount of WX-UK1 or WX-671 used to practice the present invention for therapeutic treatment of conditions caused by a disease varies depending upon the manner of administration, the age, body weight, and general health of the patient. Ultimately, the prescribers will decide the appropriate amount and dosage regimen.

By means of the present invention, one possibility for trypsin inhibition in living beings, in particular man, is provided by administration of an efficacious amount of the compounds according to the invention. The dose to be administered depends on the nature and severity of the diseases to be treated. In an embodiment, the daily dose is in the range from 0.01-100 mg/kg of active substance per body weight. In an embodiment, the daily dose is in the range from 0.1-50 mg/kg of active substance per body weight. In an embodiment, the daily dose is in the range from 0.5-40 mg/kg of active substance per body weight. In an embodiment, the daily dose is in the range from 1-30 mg/kg of active substance per body weight. In an embodiment, the daily dose is in the range from 5-25 mg/kg of active substance per body weight.

As used herein, a “dosage form” is a form intended for oral, subcutaneous, intraveneous or inhaled administration. A “solid oral dosage form” includes, but is not limited to, tablets, capsules, granules and powders. An “inhaled dosage form” includes, but is not limited to inhalers and nebulizers.

As used herein, the term “inhibitor” refers to a molecule that affects the activity of enzymes. The inhibitors of the present invention are reversible meaning they form weak interactions with their target enzyme and are easily removed. A reversible inhibitor forms a transient interaction with an enzyme. The strength of the binding between an enzyme and a reversible inhibitor is defined by the dissociation constant (K_{d}). The smaller the value of K_{d} the stronger the interaction between the enzyme and inhibitor and the greater the inhibitory effect. When talking about enzyme inhibition K_{d} is referred to as Kᵢ.

As used herein, the term “digestive disease” refers to any disease which involves the gastrointestinal tract or the accessory digestive organs (the liver, pancreas, and gallbladder). Examples of digestive diseases of the present disclosure include, but are not limited to, irritable bowel syndrome (IBS), inflammatory bowel disease (IBD), gastritis and pancreatitis. In an embodiment, a patient having an inflammatory digestive disease has increased/upregulated trypsin-like activity, as determined by serum levels of trypsin. In an embodiment, the increased trypsin-like activity is an upregulation of trypsin-2. In an embodiment, the increased trypsin-like activity is an upregulation of trypsin-3.

As used herein, the term “in situ zymography” refers to a technique that measured the proteolytic activity of a protease using a substrate specific for the protease assayed that is modified so that upon digestion it is readily visible. The substrate is placed on a tissue section, then the sample is incubated at an optimal temperature in a humidity chamber, which allows digestion of the substrate by the activated enzyme in its native location. For tissue fixation, unfixed frozen tissue (cryosections) or fixatives based on ethanol or zinc can be used. A few different approaches can be used for visualization. For example, the effects of WX-UK1 incubation on human colonic tissue samples can be measured by in situ zymography. Examples of human colonic tissue includes those obtained from patients with IBS, defined using the Rome III or Rome IV criteria, for example, and those obtained from healthy controls.

As used herein, the term “increased/upregulated trypsin-like activity” refers to mean trypsin levels markedly raised above the upper limit of normality. In an embodiment, the mean trypsin in a patient having a digestive disease is significantly higher than the upper limit of normality (P less than 0.001). In an embodiment, the mean trypsin levels are detected in serum.

By means of the present invention, one possibility for trypsin inhibition in living beings, in particular man, is provided by administration of an efficacious amount of a compound according to the invention. The dose to be administered depends on the nature and severity of the diseases to be treated. In an embodiment, the daily dose is in the range from 0.01-100 mg/kg of active substance per body weight, more highly preferably 0.1-50 mg/kg, more highly preferably 0.5-40 mg/kg, more highly preferably 1-30 mg/kg, more highly preferably 5-25 mg/kg.

### WX-UK1 and WX-671

As used herein, the term WX-UK1 refers to a synthetic small molecule inhibitor of serine proteases, shown in Formula I below, including stereoisomers, racemic mixtures, metabolites, pharmaceutically acceptable salts, crystals, or any combination thereof.

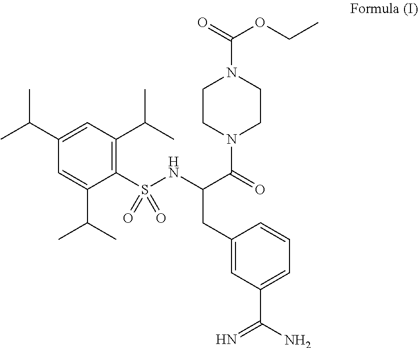 [SMILES: CCOC(=O)N1CCN(C(=O)C(Cc2cccc(C(=N)N)c2)NS(=O)(=O)c2c(C(C)C)cc(C(C)C)cc2C(C)C)CC1]

WX-UK1 can be present as racemates as well as compounds in L- or D-form. In an embodiment, the WX-UK1 used in the methods of the present invention are compounds in L-form only. In an embodiment, WX-UK1 is present as a salt with inorganic acids, preferably as hydrochlorides, or as a salt with appropriate organic acid. In an embodiment, WX-UK1 can be used together with at least one appropriate pharmaceutical additive for the preparation of orally, subcutaneously or intravenously administrable drugs.

As used herein, the term WX-671 refers to the oral prodrug of WX-UK1, shown in Formula II below, including stereoisomers, racemic mixtures, metabolites, pharmaceutically acceptable salts, crystals, or any combination thereof. The chemical name of WX-671 is N-α-(2,4,6-triisopropylphenylsulfonyl)-3-hydroxyamidino-(L)-phenylalanine-4-ethoxycarbonylpiperazide.

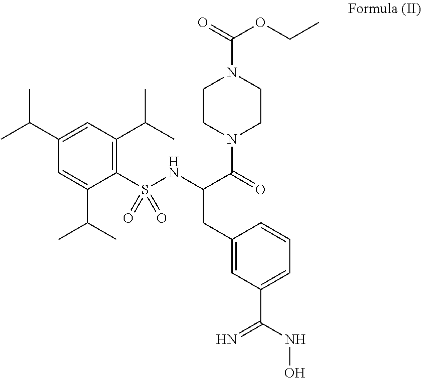 [SMILES: CCOC(=O)N1CCN(C(=O)C(Cc2cccc(C(=N)NO)c2)NS(=O)(=O)c2c(C(C)C)cc(C(C)C)cc2C(C)C)CC1]

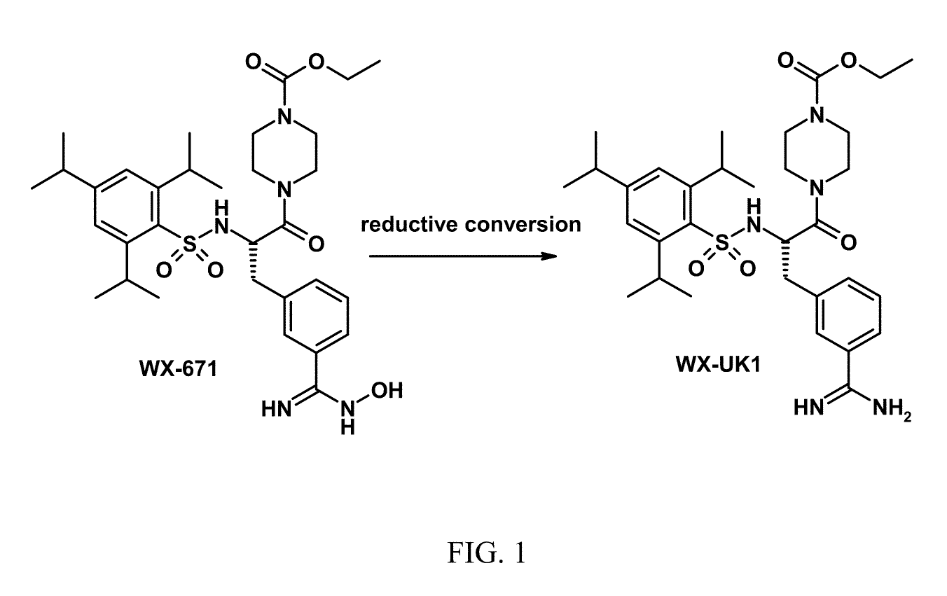

WX-671 can be present as powders, salts and crystalline forms thereof. The compounds according to the invention can optionally be used with suitable pharmaceutical excipients and/or vehicles for the production of medicaments and can be prepared in a suitable pharmaceutical formulation, for example as tablets, coated tablets, capsules, pastilles, powder, syrup, suspension, solution or the like. FIG. 1 shows reductive conversion of the prodrug WX-671 to pharmacologically active WX-UK1.

In an embodiment, WX-671 is available as a hydrogen sulphate salt designated as “WX-671.1” or “upamostat hydrogen sulphate” or “formula (III)” herein:

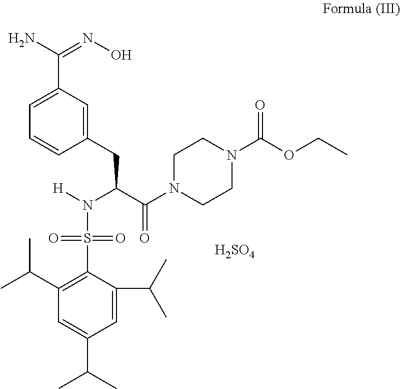

The molecular formula for WX-671.1 is C₃₂H₄₇N₅O₆SxH₂SO₄ and it is a non-hygroscopic, white to yellowish powder which is freely soluble in dimethyl sulfoxide and soluble in ethanol having a relative molecular mass of 727.91 g/mol (MW free base: 629.83 g/mol). The drug substance is very slightly soluble in water or 0.1 M HCl and can be filled in hard gelatin capsules (Ph. Eur.).

Previously, in preclinical animal tumor models, parenterally administered WX-UK1 had been shown to reduce the growth rate of implanted tumors, to inhibit their invasion into nearby lymph nodes and their spreading to distant target organs of metastasis. In the same animal tumor models oral administration of the prodrug WX-671 was similarly effective at reducing tumor-related endpoints as parenteral WX-UK1. As a prodrug, WX-671 is inactive as a serine protease inhibitor in-vitro and is activated to WX-UK1 after intestinal absorption. Previously, it was shown that the active metabolite WX-UK1 inhibited urokinase type plasminogen activator (uPA) and plasmin with Kᵢ values of 0.65-0.9 μM and 1.46-2.4 μM, respectively. In addition, WX-UK1 inhibited bovine trypsin with a Kᵢ value of 0.037 μM. As discussed below, just because WX-UK1 had a Kᵢ value of 0.037 μM does not mean that the specificity (Kᵢ value) would be the same for other mammals, such as human trypsin.

According to aspects illustrated herein, the inhibitory constant (Kᵢ-value) between a human serine protease and WX-UK1 is less than or equal to 0.25 μM, less than or equal to 0.20 μM, less than or equal to 0.15 μM, and less than or equal to 0.10 μM. In an embodiment, the Kᵢ-value between human trypsin-1 and WX-UK1 is between 0.50 μM and 0.10 μM, between 0.30 μM and 0.2 μM, about 0.19±0.01 μM. In an embodiment, the Kᵢ-value between human trypsin-2 and WX-UK1 is between 0.050 μM and 0.010 μM, between 0.60 μM and 0.090 μM, between 0.070 μM and between 0.080 μM, about 0.075±0.003 μM. In an embodiment, the Kᵢ-value between human trypsin-3 and WX-UK1 is between 0.005 μM and 0.030 μM, between 0.010 μM and 0.025 μM, between 0.015 μM and 0.020 μM, about 0.019±0.004 μM. In an embodiment, the Kᵢ-value between human MT-SP1/matriptase and WX-UK1 is between 0.50 μM and 0.10 μM, between 0.40 μM and 0.3 μM, about 0.20±0.01 μM.

### Serine Proteases

Serine proteases are present in virtually all organisms and function both inside and outside the cell; they exist as two families, the ‘trypsin-like’ and the ‘subtilisin-like’. The mammalian serine proteases have a common 3D structure, although there are quite significant differences in some regions of the surface, reflecting the different physiological functions, and resulting in different interactions with different molecules.

Trypsins vary greatly among mammals, and as such, bovine trypsin is a serine protease homolog (SPH) to human trypsin 1, but not the same. Trypsins between different mammals are not alike. For example, mice have 8 trypsins, none like any of the 6 human trypsins.

Unexpectedly, it has now been found that WX-UK1, which up until this time was known as a potential anti-tumor, anti-metastasis agent for therapeutic use against solid tumors, has potential use in the treatment of trypsin driven inflammatory diseases, including, but not limited to, pancreatitis, gastritis, inflammatory bowel disease, irritable bowel syndrome, inflammatory lung diseases, and inflammatory blood vessel disease all non-cancer indications.

It has now been discovered that the rate of association between WX-UK1 and trypsin-2 is faster than the rate of association between WX-UK1 and uPA, a previously known target of WX-UK1. Further, it has now been discovered, through drug-binding kinetic studies, that the rate of dissociation between WX-UK1 and trypsin-2 is slower than the rate of association between WX-UK1 and uPA.

It has now been discovered that the rate of association between WX-UK1 and trypsin-3 is faster than the rate of association between WX-UK1 and uPA, a previously known target of WX-UK1. Further, it has now been discovered, through drug-binding kinetic studies, that the rate of dissociation between WX-UK1 and trypsin-3 is slower than the rate of association between WX-UK1 and uPA.

In an embodiment, such dissociation characteristics are likely to offer greater control over the issue of selectivity and toxicity, as slow dissociation is often associated with high binding affinity, and WX-UK1 is likely to display pronounced target selectivity and a broad therapeutic window. Conversely, because of its potential lower affinity for collateral targets, WX-UK1 is also likely to dissociate faster from undesirable, adverse event-mediating ones. In such case, the drug-primary target complexes will last longer than the undesirable complexes so that the beneficial effects of the drug are likely to outlast the adverse events.

By discovering that trypsin-2 and tryspin-3 are high affinity targets for WX-UK1, it is believed that lower doses of WX-UK1 or its prodrug WX-671 may be needed to treat a trypsin driven inflammatory disease as compared with the doses of WX-UK1 (or its prodrug WX-671) that have been taught for treating cancers.

In an embodiment, when treating a patient for a trypsin driven inflammatory disease, WX-671 is co-administered with other pharmaceuticals to enhance therapeutic efficacy. In an embodiment, when treating a patient for a trypsin driven inflammatory disease, WX-UK1 is co-administered with other pharmaceuticals to enhance therapeutic efficacy.

### Human Trypsin-2 and Human Trypsin-3

Trypsin is usually considered as a digestive enzyme released in the upper gastrointestinal tract. Three isoforms of trypsin have been cloned from the human pancreas: PRSS1 for cationic trypsin, PRSS2 for anionic trypsin, and PRSS3 for mesotrypsin. Although traditionally considered as pancreatic enzymes, the different forms of trypsin can also be released in other tissues, synthesized by a number of different cell types, such as neurons, epithelial and endothelial cells. In particular, mesotrypsin is expressed in epithelial cell lines derived from normal colonic mucosa (NCM-460). A significant increase in trypsin-like activity was detected in colonic lumenal washes and colonic tissues from animal models of inflammatory bowel disease (IBD). Data collected from IBD patients tissue biopsies showed an increase serine protease activity similar to trypsin-like enzymes, compared to the activity detected in healthy individuals. These results strongly suggest that uncontrolled increase of intestinal trypsin activity takes place in chronically inflamed gut and might play a role in the pathogenesis of IBD. Further, premature activation of trypsin in the pancreas results in self-destruction through autodigestion and thus pancreatitis. Trypsin is a potent activator of proteinase-activated receptor 2 (PAR₂), a G protein—coupled receptor that is widely distributed throughout the body and believed to play an important role in inflammation. PARs are characterized by a unique activation mechanism involving the proteolytic unmasking of a tethered ligand that stimulates the receptor.

### Human Membrane-Type-Serine-Protease 1 (MT-SP1)/Matriptase

Unexpectedly, it has now been found that WX-UK1, which up until this time was known as a potential anti-tumor, anti-metastasis agent for therapeutic use against solid tumors, has potential use in the treatment of MT-SP1/matriptase driven inflammatory diseases, including, but not limited to, inflammatory skin diseases and inflammatory blood vessel diseases, both non-cancer indications.

It has now been discovered that the rate of association between WX-UK1 and matriptase is faster than the rate of association between WX-UK1 and uPA, a previously known target of WX-UK1. Further, it has now been discovered, through drug-binding kinetic studies, that the rate of dissociation between WX-UK1 and matriptase is slower than the rate of association between WX-UK1 and uPA.

In an embodiment, such dissociation characteristics are likely to offer greater control over the issue of selectivity and toxicity, as slow dissociation is often associated with high binding affinity, and WX-UK1 is likely to display pronounced target selectivity and a broad therapeutic window. Conversely, because of its potential lower affinity for collateral targets, WX-UK1 is also likely to dissociate faster from undesirable, adverse event-mediating ones. In such case, the drug-primary target complexes will last longer than the undesirable complexes so that the beneficial effects of the drug are likely to outlast the adverse events.

By discovering that matriptase is a high affinity target for WX-UK1, it is believed that lower doses of WX-UK1 or its prodrug WX-671 may be needed to treat a matriptase driven inflammatory disease as compared with the doses of WX-UK1 (or its prodrug WX-671) that have been taught for treating cancers.

MT-SP1/matriptase is a member of the type II transmembrane serine protease (TTSP) family, having the broadest expression pattern of all TTSPs, being detected in a wide range of human tissues. It has been shown that MT-SP1/matriptase connects the coagulation cascade to epithelial signaling and proteolysis. The link between matriptase and coagulation initiation could contribute to the pathogenic effects of extrinsic pathway activation in inflammation. Studies have shown that matriptase is activated in response to inflammatory stimuli. Matriptase cleaves and activates the proforms of urokinase-type plasminogen activator (uPA), hepatocyte growth factor (HGF), and PAR₂.

Matriptase and its inhibitor hepatocyte growth factor activator inhibitor (HAI-1) are required for normal epidermal barrier function, and matriptase activity is tightly regulated during the process. Given the role that matriptase plays in skin barrier formation and the extremely tight regulation of matriptase activity, inappropriate elevation may have an adverse impact on epidermal differentiation and enhance the disease phenotype or prolong the recovery from skin diseases. Many skin diseases are accompanied by inflammation associated with recruitment of inflammatory leukocytes leading to cytokine release.

Further, recent studies have found that, dependent on its proteolytic activity, MT-SP1/matriptase stimulates de novo synthesis of the proinflammatory cytokines IL-8 and IL-6 through activation of PAR₂ in endothelial cells. Further, PAR₂ has been identified as the mediator of MT-SP1/matriptase-induced IL-8 expression. MT-SP1/matriptase is expressed in monocytes, thus, interaction of monocytic MT-SP1/matriptase with endothelieal PAR₂ may contribute to inflammatory blood vessel diseases, including, but not limited to, arteritis and atherosclerosis.

Most-likely, matriptase is involved in several physiological and pathophysiological functions, including, but not limited to, skin disorders, arteritis, atherosclerosis, osteoarthritis and in the activation of certain viral surface glycoproteins.

### Compounds of the Present Invention for Treating Irritable Bowel Syndrome

Irritable bowel syndrome (“IBS”) refers to a functional gastrointestinal disorder with a group of symptoms, including abdominal pain and changes in the pattern of bowel movements without any evidence of underlying damage. Trypsin-3 has been shown to be upregulated in stimulated intestinal epithelial cells and in tissues from patients with IBS. This upregulation causes signaling to human submucosal enteric neurons and mouse sensory neurons, and to induce visceral hypersensitivity in vivo, all by a protease-activated receptor-2 dependent mechanism. Trypsin-3 could be used as a marker of epithelial dysfunction in patients with IBS. In an embodiment, an effective amount of WX-UK1 or its prodrug WX-671 is administered over a suitable period of time to a patient having irritable bowel syndrome. In an embodiment, the WX-UK1 or its prodrug WX-671 is administered alone as a standalone therapy. In an embodiment, the WX-UK1 or its prodrug WX-671 is co-administered with another drug.

### Compounds of the Present Invention for Treating Pancreatitis

Pancreatic digestive enzymes are stored as inactivated precursors in pancreatic acinar cells, and are normally only activated after they reach the small intestine. Under normal conditions, digestive enzyme activation is strictly controlled. Trypsinogens are produced at high concentrations by the pancreas. After secretion into the gastrointestinal tract, they are activated to trypsins by enterokinase. Trypsins are major digestive enzymes, and they further activate other pancreatic enzymes. Acinar cells in the pancreas synthesize and secrete the trypsin inhibitor serine protease inhibitor Kazal type 1 (SPINK1) that acts as a safeguard against trypsin activation within the pancreas. Trypsinogen 3 is a minor trypsinogen isoform in the pancreas. In contrast with trypsin 1 and 2, trypsin 3 degrades pancreatic SPINK1, which may lead to an excess of active trypsin and development of pancreatitis. Trypsinogen activation is one factor that determines the severity of pancreatitis. In an embodiment, an effective amount of WX-UK1 or its prodrug WX-671 is administered over a suitable period of time to a patient having pancreatitis. In an embodiment, the WX-UK1 or its prodrug WX-671 is administered alone as a standalone therapy. In an embodiment, the WX-UK1 or its prodrug WX-671 is co-administered with another drug.

### Compounds of the Present Invention for Treating Lung Injury

In an embodiment, an effective amount of WX-UK1 or its prodrug WX-671 is administered over a suitable period of time is able to stop or decrease symptoms associated with lung injury in an animal. Without being bound by theory, trypsin may induce pulmonary vascular injury by activating the complement system and by generating leukocyte activation. Activated leukocytes and their secretory products, including oxygen radicals, proteases, and cytokines, may subsequently be critical in tissue destruction. Trypsin and activation of circulating trypsinogen contribute to pancreatitis-associated lung injury (PALI). Acute lung injury (ALI) is a condition in which fluid builds up in the lungs. That makes it difficult to get enough oxygen into the bloodstream and to vital organs. Acute respiratory distress syndrome (ARDS) is an example of an ALI, characterized by widespread inflammation in the lungs. In the lung, trypsin may induce tissue injury not only through initiating matrix-degrading proteolytic cascade, but also through proinflammatory actions. Activated trypsin causes damage to pulmonary vasculature and increases endothelial permeability. In previous laboratory experiments, trypsin has been shown to cause leukostasis in the pulmonary vasculature and thus activated trypsin could intensify intravascular coagulation in the pulmonary microcirculation. In post mortem studies, patients with acute pancreatitis have been shown to have intravascular fibrin thrombi in different tissues, including the lungs. Trypsin is capable of activating different complement factors directly, which can stimulate cytolysis and chemotactic leukocytes. Complement has been shown to produce ARDS raising the possibility of developing respiratory insufficiency. In an embodiment, an effective amount of WX-UK1 or its prodrug WX-671 is administered over a suitable period of time to a patient having a lung injury. In an embodiment, the WX-UK1 or its prodrug WX-671 is administered alone as a standalone therapy. In an embodiment, the WX-UK1 or its prodrug WX-671 is co-administered with another drug.

The effectiveness of compounds of the present invention as inhibitors of human serine proteases can be determined using a relevant purified serine protease, and an appropriate synthetic substrate. The rate of hydrolysis of the chromogenic or fluorogenic substrate by the relevant serine protease can be measured both in the absence and presence of compounds of the present invention. Assays may be conducted at room temperature or at 37° C. Hydrolysis of the substrate results in release of amino trifluoromethylcoumarin (AFC), which was monitored spectrofluorometrically by measuring the increase in emission at 510 nm with excitation at 405 nm. A decrease in the rate of fluorescence change in the presence of inhibitor is indicative of enzyme inhibition. Such methods are known to one skilled in the art. The results of this assay are expressed as the inhibitory constant, Kᵢ.

In an embodiment, WX-UK1 may be administered intravenously to a patient at dosages from about 0.1 mg/kg to about 4.0 mg/kg, from about 0.3 mg/kg to about 3.5 mg/kg, from about 0.6 mg/kg to about 2.8 mg/kg, from about 1.0 mg/kg to about 2.1 mg/kg, and from about 1.1 mg/kg to about 1.6 mg/kg, for an 80 kg patient. Intravenous infusions may be administered via a central or peripheral catheter. A total volume of 1000 ml may be administered over 2 hours at a rate of 500 ml/h by means of an infusion pump. In an embodiment, WX-UK1 infusion may be given once a week for a suitable period of time. In an embodiment, WX-UK1 infusion may be given twice a week for a suitable period of time. In an embodiment, WX-UK1 infusion may be given three times a week for a suitable period of time. In an embodiment, WX-UK1 infusion may be given four times a week for a suitable period of time. In an embodiment, WX-UK1 infusion may be given five times a week for a suitable period of time.

According to aspects illustrated herein, high affinity targets for the compound N-α-(2,4,6-triisopropylphenylsulfonyl)-3-amidino-(L)-phenylalanine 4-ethoxycarbonylpiperazide (“WX-UK1”) have been identified with important ramifications in health and disease and which indicates an important usage for WX-UK1 in the treatment of human inflammatory blood vessel disease. Prior to the filing date of this application, WX-UK1 was disclosed for administration in the treatment of, inter alia, cancers. However, WX-UK1 was not known for the treatment of vasculitis, including, but not limited to, arteritis and atherosclerosis. In an embodiment, WX-UK1 or its prodrug WX-671, as detailed below, can be used for the treatment of arteritis. In an embodiment, WX-UK1 or its prodrug WX-671, can be used for the treatment of atherosclerosis.

PAR₂ is activated by pro-inflammatory proteases such as trypsin and also by the TF-dependent FVII/Xa/matriptase pathway. In an embodiment, it is believed that WX-UK1 inhibits human trypsin and human matriptase from cleaving the extracellular N-terminal domain of human protease activated receptor-2 (PAR₂), and thus PAR₂ cannot be activated. In an embodiment, by blocking activation of PAR₂, the inflammatory process is reduced or stopped. In an embodiment, it is believed that WX-UK1 inhibits human trypsin and human matriptase and in turn blocks a PAR₂-dependent mechanism that is responsible for signaling sensory neurons, such as primary afferent nerves and submucosal enteric neurons, thus resulting in a decrease in neurogenic inflammation and pain. According to aspects illustrated herein, WX-UK1 or its prodrug WX-671 can be used as a therapeutic active compound for the treatment of PAR₂ disorders.

According to aspects illustrated herein, high affinity targets for the compound N-α-(2,4,6-triisopropylphenylsulfonyl)-3-amidino-(L)-phenylalanine 4-ethoxycarbonylpiperazide (“WX-UK1”) have been identified with important ramifications in health and disease and which indicates an important usage for WX-UK1 in the treatment of human alpha-1 antitrypsin deficiency (A1AD). A1AD is a genetic disorder that causes defective production of alpha-1 antitrypsin (A1AT), the most abundant human serine protease inhibitor, leading to decreased A1AT activity in the blood and lungs, and deposition of excessive abnormal A1AT protein in liver cells. Prior to the filing date of this application, WX-UK1 was disclosed for administration in the treatment of, inter alia, cancers. However, WX-UK1 was not known for the treatment of A1AD. In an embodiment, WX-UK1 or its prodrug WX-671, can be used for the treatment of human A1AD in which there is an unmet medical need.

In an embodiment, a kit for use with a patient needing a trypsin-3 inhibitor to treat a disease comprises one or more containers comprising a plurality of solid forms of N-α-(2,4,6-triisopropylphenylsulfonyl)-3-hydroxyamidino-(L)-phenylalanine 4-ethoxycarbonylpiperazide (“WX-671”), wherein the solid forms comprise a therapeutically effective amount of WX-671 in order to treat the patient. In an embodiment, the solid forms are part of a pharmaceutical composition further comprising a pharmaceutically acceptable excipient or carrier. In an embodiment, the pharmaceutical composition is a single unit dosage form. In an embodiment, the pharmaceutical composition is a tablet. In an embodiment, the pharmaceutical composition is a capsule.

In an embodiment, a kit for use with a patient needing a trypsin-2 inhibitor to treat a disease comprises one or more containers comprising a plurality of solid forms of N-α-(2,4,6-triisopropylphenylsulfonyl)-3-hydroxyamidino-(L)-phenylalanine 4-ethoxycarbonylpiperazide (“WX-671”), wherein the solid forms comprise a therapeutically effective amount of WX-671 in order to treat the patient. In an embodiment, the solid forms are part of a pharmaceutical composition further comprising a pharmaceutically acceptable excipient or carrier. In an embodiment, the pharmaceutical composition is a single unit dosage form. In an embodiment, the pharmaceutical composition is a tablet. In an embodiment, the pharmaceutical composition is a capsule.

### EXAMPLES

### Example 1

### Bioinformatics Analysis

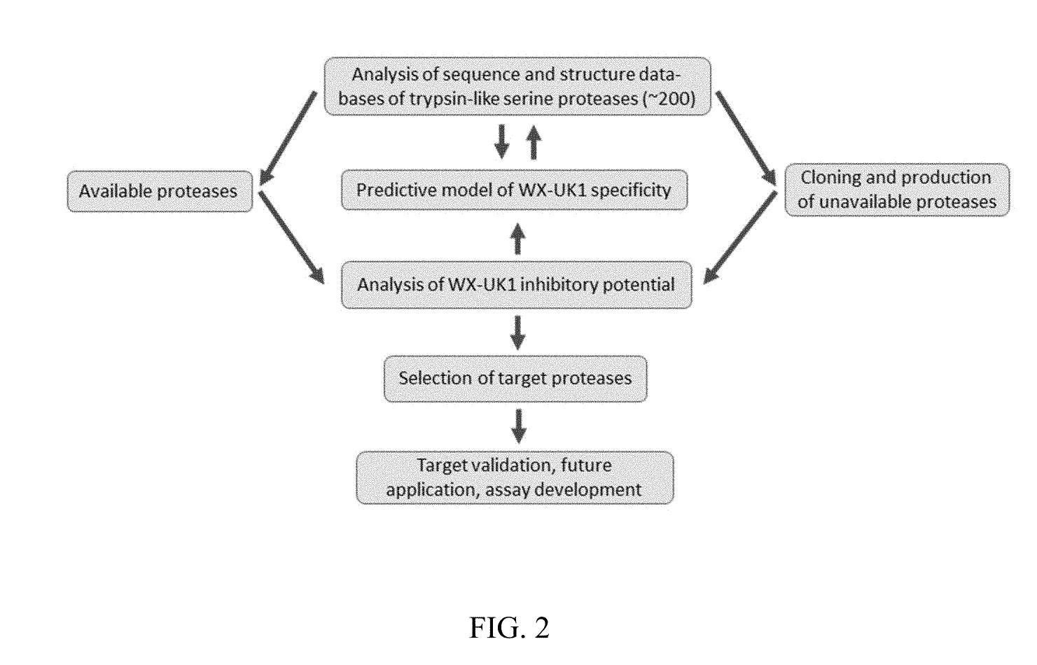

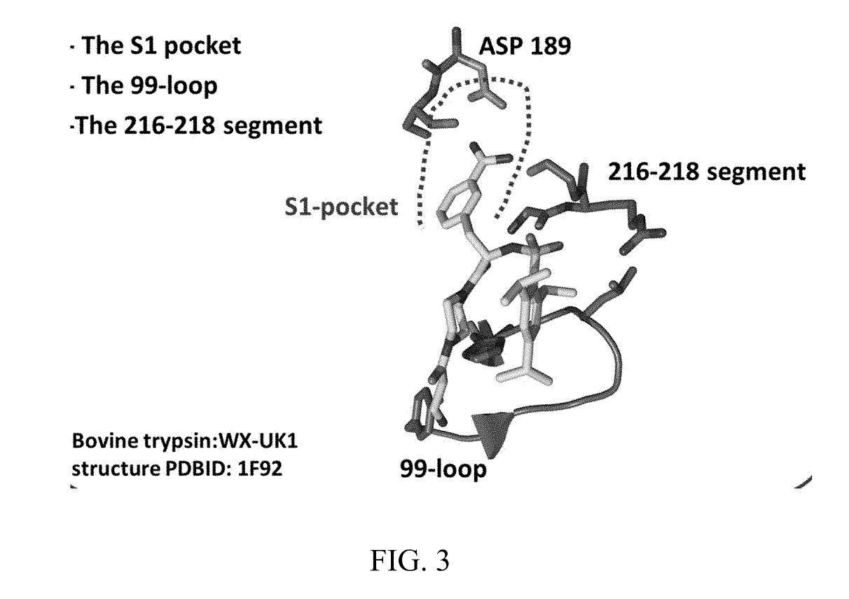

As schematically illustrated in FIG. 2, the starting point of the selection process was ˜200 trypsin-like serine proteases of the human proteasome, most of which play crucial roles in homeostasis and diseases, and all potential targets with varying potency for inhibition by WX-UK1. Application of the iterative process resulted in a progressively smaller selection of potential high-affinity targets. By a thorough analysis of the sequence database and available structural information of all human chymotrypsin-like serine proteases with trypsin-like specificity, a circular iterative process was carried out in which structure and sequence based criteria for WX-UK1 inhibition was tested by measuring WX-UK1 inhibition of selected proteases, key structural components were elucidated as shown in FIG. 3. Among these were selected a subset for biochemical analysis based on a ranking of likely binding from inspection of calculated 3D structure models of possible WX-UK1:protease complexes and sequence alignment focusing on residues in interaction surfaces. Samples of the selected proteases were prepared and characterized biochemically with respect to inhibitory constant (Kᵢ) of inhibition by WX-UK1-1 and affinity (K_{d}) of the interaction. The Kᵢ values were determined from series of enzymatic activity assays where the protease activity was monitored by the turnover of chromogenic substrates at 37° C. in the presence of albumin, in the presence of increasing WX-UK1 concentration. When possible, the dissociation constant K_{d} of binding was determined using state-of-the-art surface plasmon resonance (BIACORE), monitoring real time kinetics of association and dissociating rate constants, all at 25° C.

### Example 2

### The Inhibitor Constant of WX-UK1 to Various Human Proteases

Kᵢ-values were determined by measuring the effect of WX-UK1 on human serine protease cleavage of chromogenic substrates. For determination of Kᵢ-values, concentration series of WX-UK1 were pre-incubated with the target human serine protease before chromogenic substrate was added to initiate the reaction. The reaction velocities were determined from the slopes using linear regression and these were normalized to that of the non-inhibited reaction. The normalized activities were plotted against WX-UK1 concentrations before the Kᵢ-values were obtained by non-linear regression using equation 1.

$$\frac{{v}_{i}}{{v}_{0}} = \frac{{K}_{i} \cdot KM + [ S ]}{( {K}_{i} \cdot [ S ] ) + KM \cdot ( {K}_{i} \cdot [ I ] )} \qquad Equation 1$$

The KM-parameter was obtained by standard Michaelis-Menten kinetics. Serine protease was added to a suitable concentration series of substrate, high enough to yield an experimental Vmax value. The subsequent reaction velocities were plotted against the substrate concentrations before the KM-value was derived using the Michaelis-Menten equation (2).

$$v = \frac{{v}_{max} \cdot [ S ]}{{K}_{M} + [ S ]} \qquad Equation 2$$

All experiments were performed in at least triplicates at 37° C. in EMS (30 mM Hepes, pH=7.4; 150 mM NaCl; 0.5% BSA). Reactions were monitored at 2 reads/min for at least 45 min at 405 nm. Since WX-UK1 was kept in 100% DMSO, an uninhibited DMSO-control was included in all experiments to exclude unwanted DMSO effects on protease activity. Due to poor solubility of WX-UK1 in water, the maximal final WX-UK1 concentration was 250 μM. Substrate concentrations were kept high in order to stay around 10% turnover per run.

Inhibition of Human Trypsin-1 with WX-UK1

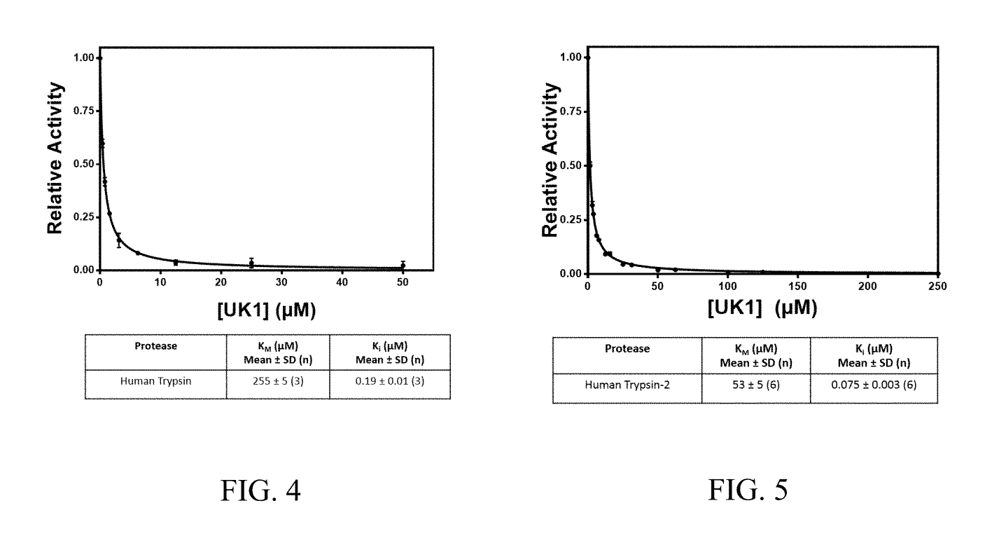

FIG. 4 is a graph of UK1 activity vs human trypsin-1 inhibitor concentration.

Inhibition of Human Trypsin-2 with WX-UK1

FIG. 5 is a graph of UK1 activity vs human trypsin-2 inhibitor concentration.

Inhibition of Human Trypsin-3 with WX-UK1

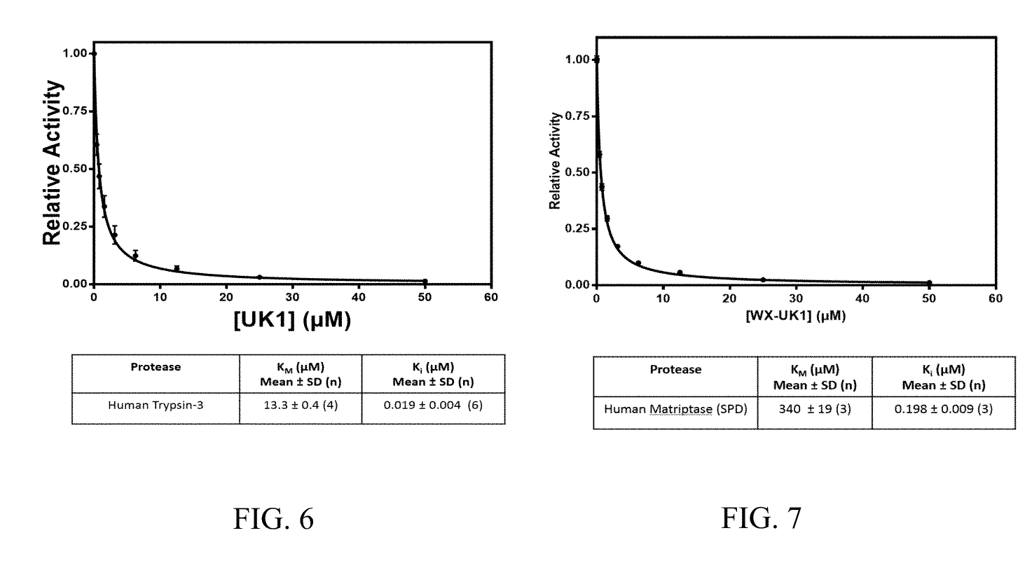

FIG. 6 is a graph of UK1 activity vs human trypsin-3 inhibitor concentration.

Inhibition of Human MT-SP1/Matriptase with WX-UK1

FIG. 7 is a graph of UK1 activity vs human matriptase (catalytic domain) inhibitor concentration

As discussed above, a bioinformatic analysis was performed of the ˜200 trypsin-like serine proteases of the human proteasome, most of which play crucial roles in homeostasis and diseases, and all potential targets for inhibition by WX-UK1. Among these we selected a subset for biochemical analysis based on a ranking of likely binding from inspection of calculated 3D structure models of possible WX-UK1:protease complexes and sequence alignment focusing on residues in interaction surfaces. The selection of the subset was narrowed down based on various criteria.

Table 1 below lists the Kᵢ values for some of the proteases in this subset to date.

TABLE 1
Kᵢ values

| | Ki (μM) | |
|---|---|---|
| Protease | Mean ± SD (n) | |
| Human Trypsin-3 | 0.019 ± 0.004 | (6) |
| Human Trypsin-2 | 0.075 ± 0.003 | (6) |
| Human Trypsin-6 | 0.10 ± 0.01 | (4) |
| Human Trypsin-1 | 0.19 ± 0.01 | (3) |
| Human Matriptase-1 | 0.20 ± 0.01 | (3) |
| Human HATL5 | 0.7 ± 0.1 | (3) |
| Human Enterokinase | 0.71 ± 0.04 | (4) |
| Human Thrombin | 0.8 ± 0.1 | (3) |
| Human uPA | 0.9 ± 0.1 | (3) |
| Human FXIa | 0.9 ± 0.1 | (6) |
| Human two-chain tPA | 1.4 ± 0.1 | (3) |
| Human HAT | 1.5 ± 0.1 | (6) |
| Human Plasmin | 2.4 ± 0.3 | (4) |
| Human FIXa | 2.5 ± 0.2 | (3) |
| Human Fxa | 2.6 ± 0.4 | (3) |
| Human C1s | 3.1 ± 0.4 | (5) |
| Human Activated Protein C | 3.9 ± 0.2 | (3) |
| Human Hepsin | 4.3 ± 0.5 | (5) |
| Human Matriptase-2 | 6.4 ± 0.3 | (4) |
| Human Spinesin | 7.7 ± 0.5 | (3) |
| Human Tryptase- [symbol] | 11 ± 2 | (3) |
| Human DESC-1 | 13 ± 2 | (3) |
| Human PRSS27 (IC50) | 19 ± 4 | (3) |
| Human Plasma Kallikrein | 26 ± 1 | (3) |
| Human HGFA | 28 ± 5 | (4) |
| Human Granzymer A | >250 | (3) |
| Human Kallikrein-8 | >250 | (3) |
| Human Kallikrein-1 | >250 | (3) |
| Human Kallikrein-11 | >250 | (3) |
| Human Prostasin (IC50) | >250 | (3) |
| Rat uPA | 0.4 ± 0.1 | (3) |
| Bovine Cationic Trypsin-1 | 0.5 ± 0.1 | (6) |
| Canine uPA | 0.7 ± 0.1 | (4) |
| Rabbit uPA | 0.8 ± 0.1 | (3) |
| Human uPA (Q192A in medium) | 2.9 ± 0.1 | (3) |
| Human uPA (H99A in medium) | 14 ± 0.4 | (3) |
| Mouse uPA | 45 ± 6 | (3) |

### Example 3

### Binding Kinetics of WX-UK1 to Various Human Proteases

Surface plasmon resonance (SPR) is the resonant oscillation of conduction electrons at the interface between negative and positive permittivity material stimulated by incident light. SPR is the basis of many standard tools for measuring adsorption of material onto planar metal (typically gold or silver) surfaces or onto the surface of metal nanoparticles. It is the fundamental principle behind many color-based biosensor applications and different lab-on-a-chip sensors. When the surface plasmon wave interacts with a local particle or irregularity, such as a rough surface, part of the energy can be re-emitted as light. This emitted light can be detected behind the metal film from various directions.

When the affinity of two ligands has to be determined, the binding constant must be determined. It is the equilibrium value for the product quotient. This value can also be found using the dynamic SPR parameters and, as in any chemical reaction, it is the association rate divided by the dissociation rate. For this, a bait ligand is immobilized on the dextran surface of the SPR crystal. Through a microflow system, a solution with the prey analyte is injected over the bait layer. As the prey analyte binds the bait ligand, an increase in SPR signal (expressed in response units, RU) is observed. After desired association time, a solution without the prey analyte (usually the buffer) is injected on the microfluidics that dissociates the bound complex between bait ligand and prey analyte. Now as the prey analyte dissociates from the bait ligand, a decrease in SPR signal (expressed in resonance units, RU) is observed. From these association (‘on rate’, kₐ) and dissociation rates (‘off rate’, k_{d}), the equilibrium dissociation constant (‘binding constant’, K_{D}) can be calculated.

The actual SPR signal can be explained by the electromagnetic ‘coupling’ of the incident light with the surface plasmon of the gold layer. This plasmon can be influenced by the layer just a few nanometer across the gold-solution interface i.e. the bait protein and possibly the prey protein. Binding makes the reflection angle change.

Biacore specializes in measuring biomolecular interactions, including protein-protein interactions, small molecule/fragment-protein interactions. Its technology is often used to measure not only binding affinities, but kinetic rate constants and thermodynamics as well. The technology is based on SPR. The SPR-based biosensors can be used in determination of active concentration as well as characterization of molecular interactions in terms of both affinity and chemical kinetics.

The following experiment was carried out to determine the binding kinetics of WX-UK1 to various serine proteases:

Buffer composition and dilution method for Biacore:

Immobilisation buffer (0.5 L):
30 mM HEPES pH 7.4
140 mM NaCl

Running buffer (0.5 L):
30 mM HEPES pH 7.4
140 mM NaCl
0.5% BSA
0.1% Tween20
0.05% DMSO (Added later)

Dilution:
50 mL running buffer is removed before addition of DMSO (called ‘-DMSO’)
225 μL DMSO is added to running buffer
50 mL running buffer with DMSO is removed (called ‘+DMSO’)
WX-UK1 stock for a concentration series of 4 μM-0 μM:
A stock of 8 mM WX-UK1 in 100% DMSO is prepared
Dilute to 4 μM using the ‘-DMSO’-stock. After this, all dilutions are made using the ‘+DMSO’-buffer

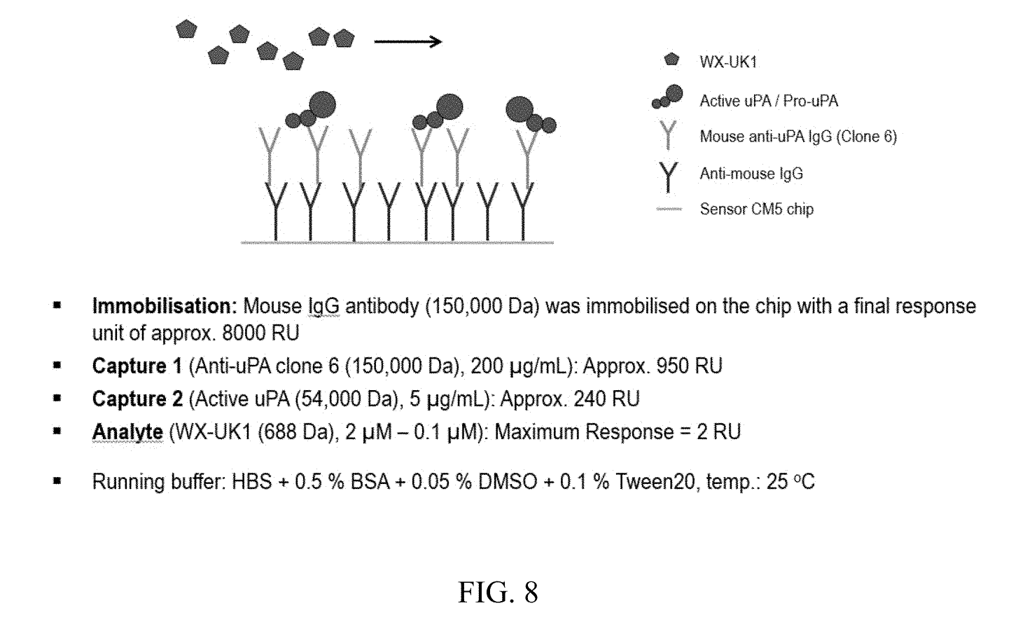

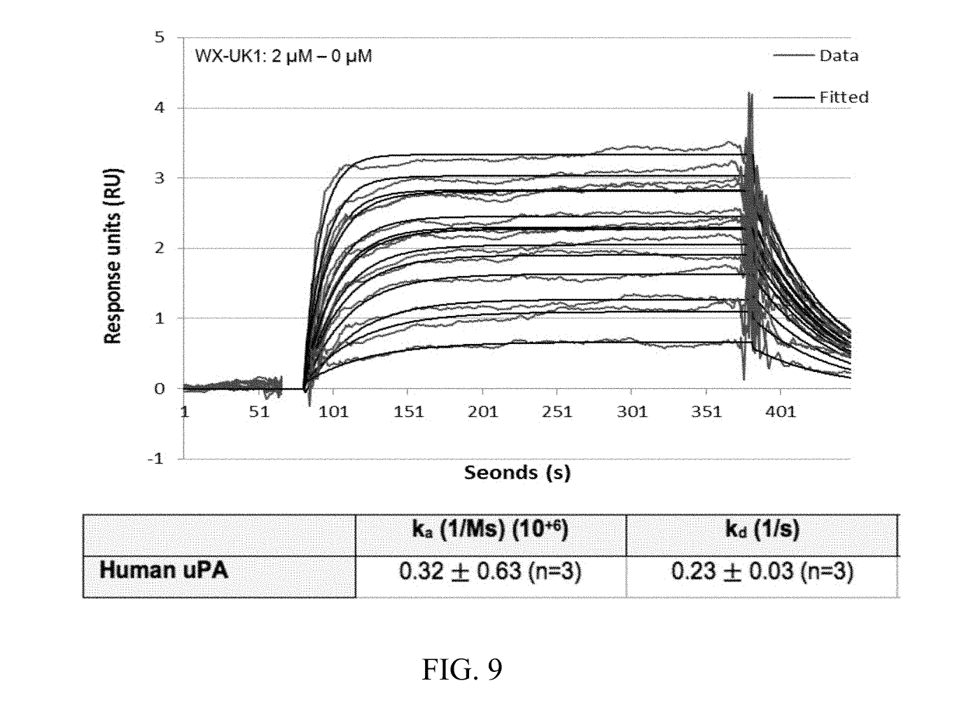

FIG. 8 is a schematic showing the setup of human uPA, and FIG. 9 shows binding kinetics of WX-UK1 binding to human uPA.

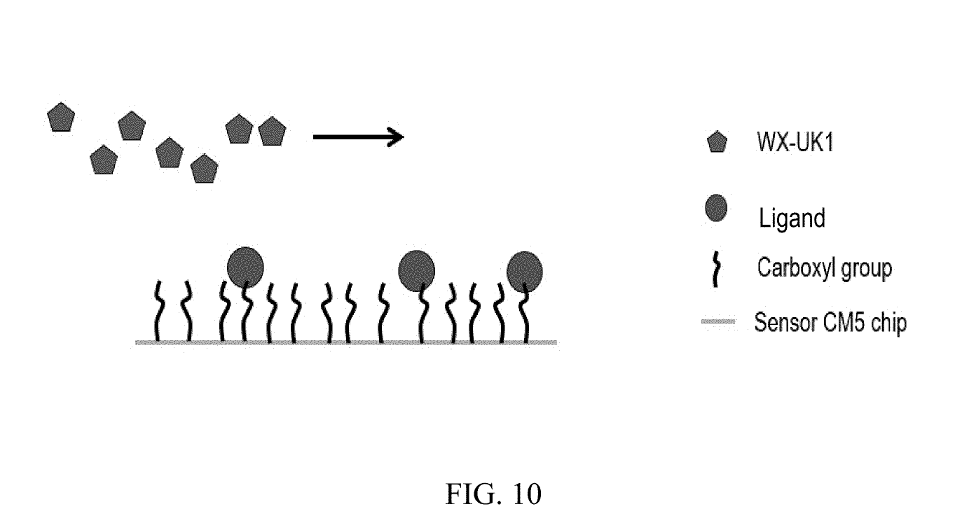

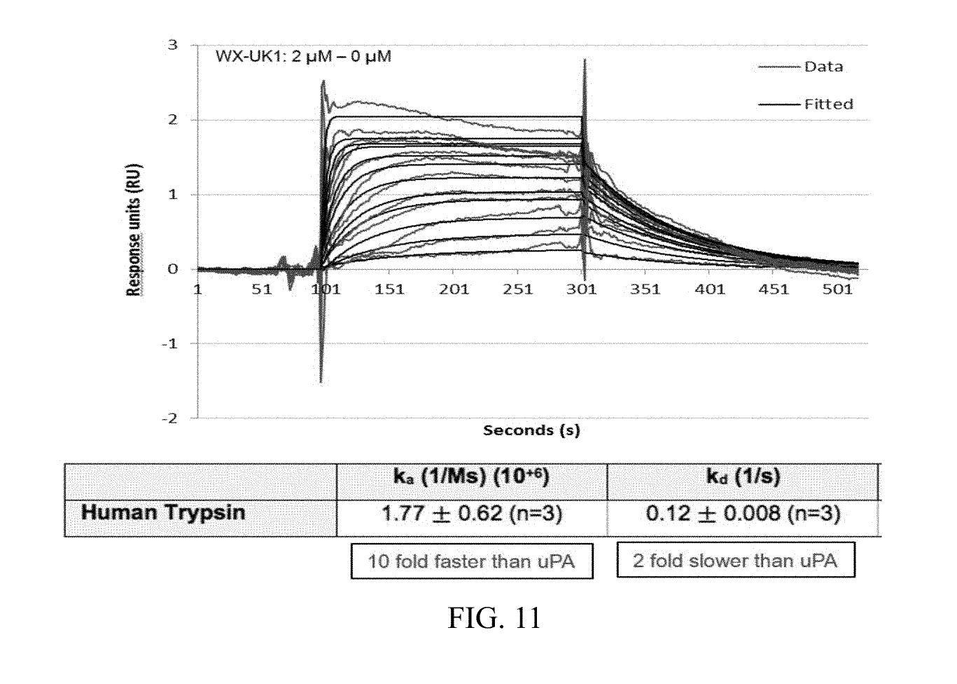

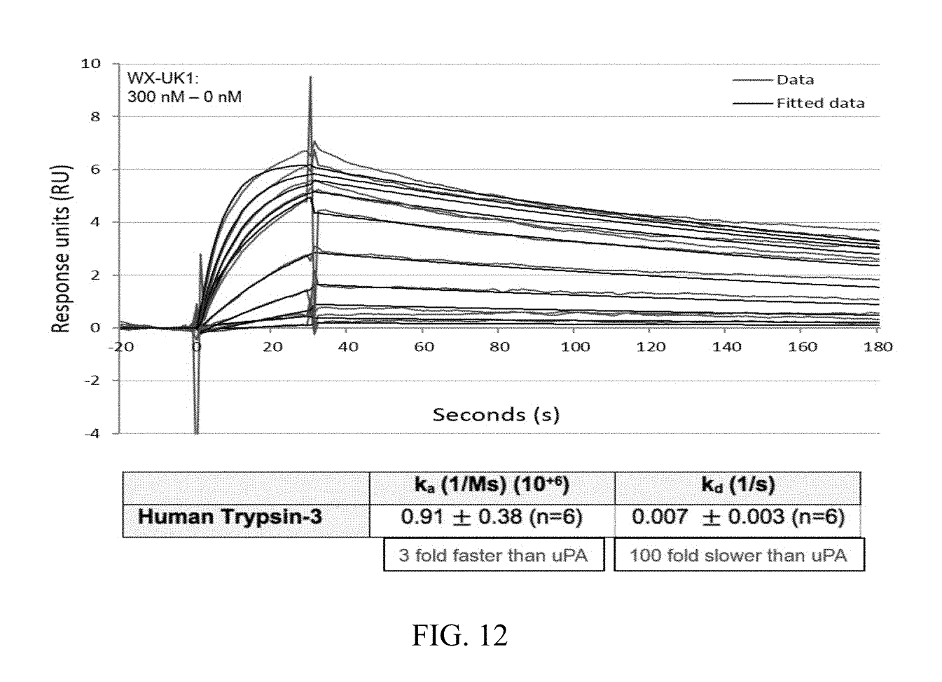

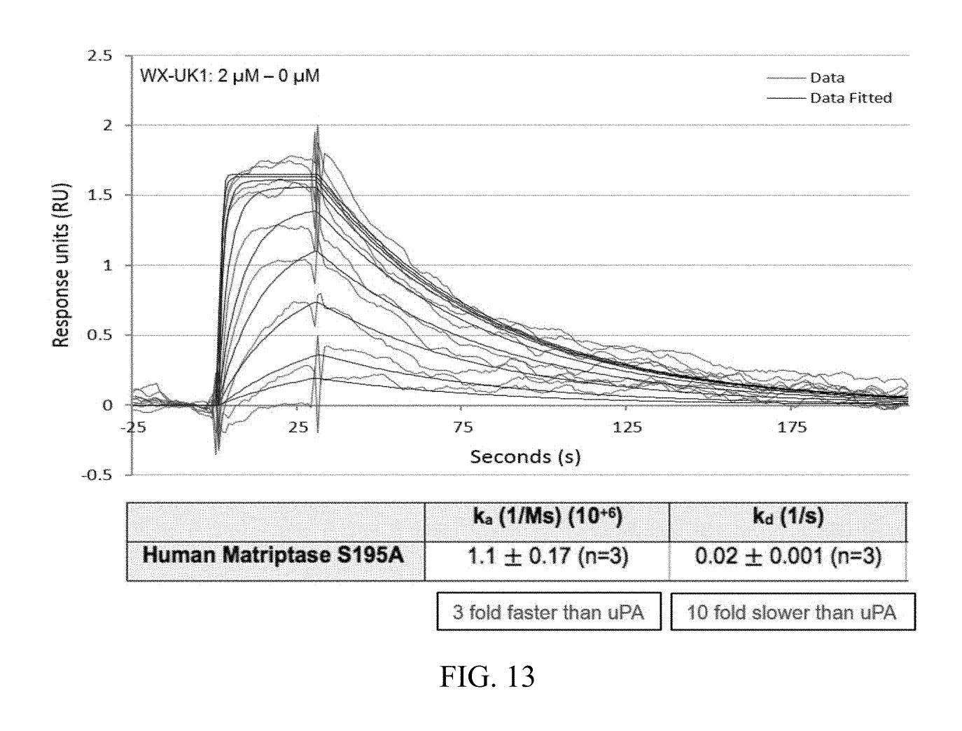

FIG. 10 is a schematic showing the setup of human trypsin-1, human trypsin-3 and human MT-SP1/matriptase. FIG. 11 shows binding kinetics of WX-UK1 binding to trypsin-1. Comparing FIGS. 9 and 11, we see that WX-UK1 associates 10 fold faster to trypsin-1 then uPA, and dissociates 2 fold slower from trypsin-1 then uPA. FIG. 12 shows binding kinetics of WX-UK1 binding to human trypsin-3. Comparison of FIGS. 9 and 12 shows that WX-UK1 associates 3 fold faster to trypsin-3 then uPA, and dissociates 100 fold slower from trypsin-3 then uPA. FIG. 13 shows binding kinetics of WX-UK1 binding to human MT-SP1/matriptase. Comparison of FIGS. 9 and 13 shows that WX-UK1 associates 3 fold faster to MT-SP1/matriptase then uPA, and dissociates 10 fold slower from MT-SP1/matriptase then uPA.

Those skilled in the art will recognize, or be able to ascertain using no more than routine experimentation, numerous equivalents to the compounds, compositions, and methods of use thereof described herein. Such equivalents are considered to be within the scope of the present disclosure and are covered by the following embodiments.

It will be appreciated by persons skilled in the art that numerous variations and/or modifications may be made to the invention as shown in the specific embodiments without departing from the scope of the invention as broadly described. The present embodiments, are, therefore, to be considered in all respects as illustrative and not restrictive.

What is claimed is:

1. A method comprising:
obtaining a biological sample from an animal having a non-cancerous medical condition;
testing the sample to obtain a trypsin concentration, wherein, if the trypsin concentration is above the upper limit of the normality, the animal is treated for a suitable period of time by administering to the animal a therapeutically effective amount of a pharmaceutical composition comprising a pharmaceutically-acceptable carrier and the compound N-α-(2,4,6-triisopropylphenylsulfonyl)-3-hydroxyamidino-phenylalanine-4-ethoxycarbonylpiperazide, the stereoisomers, racemic mixtures, metabolite, pharmaceutically acceptable salt, crystal, or any combination thereof.

2. The method of claim 1, wherein the biological sample is selected from the group consisting of blood, serum, urine, saliva, duodenal fluid and intestinal mucosal biopsies.

3. The method of claim 1, wherein the non-cancerous medical condition is selected from the group consisting of pancreatitis, gastritis, irritable bowel syndrome, and inflammatory bowel disease.

4. The method of claim 3, wherein the non-cancerous medical condition is irritable bowel syndrome.

5. The method of claim 4, wherein the irritable bowel syndrome is constipation-predominant irritable bowel syndrome.

6. The method of claim 4, wherein the irritable bowel syndrome is diarrhea-predominant irritable bowel syndrome.

7. The method of claim 3, wherein the non-cancerous medical condition is inflammatory bowel disease.

8. The method of claim 7, wherein the inflammatory bowel disease is Crohn's disease.

9. The method of claim 7, wherein the inflammatory bowel disease is ulcerative colitis.

10. The method of claim 1, wherein the non-cancerous medical condition is acute pancreatitis.

11. The method of claim 1, wherein the pharmaceutical composition is in an orally administrable form.

12. The method of claim 1, wherein the compound is present as a sulfate or hydrogen sulfate salt.

13. The method of claim 1, wherein the compound is selected from the group consisting of:
N-α-(2,4,6-triisopropylphenylsulfonyl)-3-hydroxyamidino-(L)phenylalanine-4-ethoxycarbonylpiperazide,
N-α-(2,4,6-triisopropylphenylsulfonyl)-3-hydroxyamidino-(D)phenylalanine-4-ethoxycarbonylpiperazide, and
N-α-(2,4,6-triisopropylphenylsulfonyl)-3-hydroxyamidino-(D,L)phenylalanine-4-ethoxycarbonylpiperazide,
or a physiologically compatible salt thereof.

14. The method of claim 13, wherein the compound is N-α-(2,4,6-triisopropylphenylsulfonyl)-3-hydroxyamidino-(L)phenylalanine-4-ethoxycarbonylpiperazide hydrogen sulfate.

15. The method of claim 13, wherein the compound is a crystalline form of N-α-(2,4,6-triisopropylphenylsulfonyl)-3-hydroxyamidino-(L)phenylalanine-4-ethoxycarbonylpiperazide or a physiologically compatible salt thereof.

16. The method of claim 1, wherein the animal is a human.

17. The method of claim 1, wherein the biological sample is tested to obtain a level of trypsin-3.

18. The method of claim 1, wherein the biological sample is tested to obtain a level of trypsin-2.

19. The method of claim 1, wherein the pharmaceutical composition is co-administered with another drug that is approved for treatment of the non-cancerous medical condition.

20. A method comprising:
obtaining a biological sample of duodenal fluid from a human having irritable bowel syndrome;
testing the sample to obtain a trypsin-3 concentration, wherein, if the trypsin-3 concentration is above the upper limit of the normality, the human is treated for a suitable period of time by administering to the human a therapeutically effective amount of a pharmaceutical composition comprising a pharmaceutically-acceptable carrier and the compound N-α-(2,4,6-triisopropylphenylsulfonyl)-3-hydroxyamidino-(L)phenylalanine-4-ethoxycarbonylpiperazide hydrogen sulfate.
